(19) United States
(12) Patent Application Publication
(10) Pub. No.: US 2026/0347292 A1
(43) Pub. Date: Nov. 5, 2026

(54) ALTERNATING LOAD OPERATION OF A METHANOL PLANT

(71) Applicant: L'Air Liquide, Société Anonyme pour I'Etude et I'Exploitation des Procédés Georges Claude, Paris (FR)

(72) Inventors: Vincent PENA, Frankfurt (DE); Tobias OELMANN, Bad Vilbel (DE); Veronika GRONEMANN, Frankfurt (DE); Michael STROZYK, Offenbach (DE)

(21) Appl. No.: 19/486,657

(22) PCT Filed: May 7, 2024
(86) PCT No.: PCT/EP2024/062648

§ 371 Date: Nov. 21, 2025

(30) Foreign Application Priority Data
Jun. 7, 2023 (EP) 23177928.1

(51) Int. Cl. C07C 29/151

(52) U.S. Cl. CPC C07C 29/151

(57) ABSTRACT

A process for synthesis of methanol, wherein a feed gas stream including at least a first reactant and a second reactant is supplied to a synthesis reactor assembly, wherein the synthesis reactor assembly produces methanol with the first reactant and the second reactant, wherein in at least one first case of a reduction in a molar flow rate of the feed gas stream a production rate of the methanol is at least temporarily reduced by reducing a proportion of the first reactant in the feed gas stream. A first reactant includes carbon dioxide and/or carbon monoxide and the second reactant includes hydrogen.

### CROSS REFERENCE TO RELATED APPLICATIONS

This application is a 371 of International Application No. PCT/EP2024/062648, filed May 7, 2024, which claims priority to European Patent Application No. 23177928.1, filed Jun. 7, 2023, the entire contents of which are incorporated herein by reference.

### BACKGROUND

The present invention relates to a process and an apparatus for synthesis of methanol, in particular in alternating load operation.

Conventional plants for synthesis of methanol are designed for continuous production at constant load. Changes in load are minimal in normal operation. This is made possible by continuous supplying with feed gas. Load changes and process adjustments between defined steady states are carried out very slowly over hours to days to protect the methanol plant and the catalyst.

Scheduled alternating load operation of the synthesis loop with a synthesis reactor assembly is generally not envisaged. Especially operation under very low partial load is uncommon. Minimum loads in production operations are typically at least 50% to 70% of plant capacity. The partial load case may be intended for longer periods with reduced gas availability over several weeks or months in winter.

Load changes and process adjustments between defined steady states are carried out very slowly over hours to days to protect the equipment and the catalysts of the methanol plant from damage.

Ordinary feed gas from industrial processes contains primarily hydrogen, carbon monoxide and carbon dioxide.

The synthesis of methanol may in principle be performed with hydrogen produced by an electrolyzer. The electrolyzer may inter alia be operated with renewable energies. This operation with renewable energies faces the challenge that hydrogen production is beset with fluctuations. Fluctuations in the amount of hydrogen produced from renewable energies depend on the available solar energy and the available wind energy for example. For example solar energy falls at night or wind power falls during a summer lull. Here, typical load changes can occur in the space of a few seconds to minutes. Changes to the loading of the electrolyzer are possible in a similar order of magnitude. However, load changes in the case of fluctuations in renewable energies and of the electrolyzer are an order of magnitude faster than the load change of the synthesis reactor assembly. Furthermore, the permissible operating range of the synthesis reactor assembly is more limited than that of the electrolyzer and the renewable energies. The synthesis reactor assembly for the synthesis of methanol is slow to respond.

In steady-state continuous operation the synthesis loop comprising the synthesis reactor assembly runs under virtually constant pressure and temperature conditions which accordingly means a uniform operating state for apparatuses, machines and the catalyst of the methanol plant. The pressure in the synthesis loop can be kept constant via a pressure control valve which releases inert components and unreacted reactants as well as unseparated products as soon as the synthesis loop pressure exceeds a particular pressure. What is challenging in fluctuating operation, i.e. in the case of non-constant supply of the reactants, is maintaining the operating pressure in the synthesis loop and allowing a high conversion of the reactants in the synthesis reactor assembly.

One way to increase the conversion of the reactants is to allow unreacted reactants to recirculate back around the synthesis loop (recycle). The recycle can also result in inert gas present in the reactant gas streams accumulating in the synthesis loop and stabilizing the pressure of the synthesis loop. At low inert gas proportions in the reactant gas streams this stabilization of the pressure can be too slow to follow the fluctuations of a second reactant stream and can therefore be inadequate or unusable. This effect is also unusable when the proportion of inerts in the reactant streams is very low or even nil.

Switching from full load in normal operation to partial load operation (reducing reactant gas streams) causes the pressure in the synthesis loop to decrease in the absence of additional measures such as for example altering the recycle flow rate. The reason for this is that the synthesis loop initially still contains reactant-rich recirculated recycle gas which reacts in the reactor and forms methanol. The methanol is separated in the separator. This results in a reduction in the amount of substance of the gas phase in the synthesis loop. The partial pressure of the gaseous reactants decreases. The pressure of the constant volume of the synthesis loop therefore also decreases. The pressure control valve cannot prevent the pressure drop since this can only release an elevated pressure by opening the valve.

If the feed gas stream falls rapidly within seconds to minutes due to fluctuations brought about by an electrolyzer operated with renewable energy for example, the pressure in the methanol reactor cannot be stabilized quickly enough. By contrast, a rapid increase in the feed gas stream may be stabilized via the pressure control valve in order not to exceed an operating pressure for a partial load or full load.

### SUMMARY

Having regard to the foregoing it is an object of the present invention to allow alternating load operation in the synthesis of methanol.

This object is achieved with the process and the apparatus according to the independent claims. Further advantageous embodiments of the invention are specified in the dependent claims. The features individually listed in the dependent claims may be combined with one another in technologically advantageous fashion and may define further embodiments of the invention. Moreover, the features specified in the claims are more particularly described and elucidated in the description, with further preferred embodiments of the invention being specified.

The present invention provides a process for synthesis of methanol, wherein a feed a gas stream comprises at least the first reactant and a second reactant and is supplied to a synthesis reactor assembly. The synthesis reactor assembly produces methanol with the first reactant and the second reactant. In at least one first case of a reduction in a molar flow rate of the feed gas stream a production rate of the methanol is at least temporarily reduced by reducing a proportion of the first reactant in the feed gas stream.

The first reactant and the second reactant may be mixed to give a feed gas stream. The feed gas stream is supplied to the synthesis reactor assembly. In the synthesis reactor assembly the first reactant and the second reactant can react to afford methanol as a product and optionally to afford one or more byproducts.

In the synthesis reactor assembly the first reactant and the second reactant are preferably gaseous.

The synthesis reactor assembly may be composed of a reactor system of suitable reactors. The synthesis reactor assembly by preference comprises one reactor, preferably two or more reactors arranged in parallel or in series. The synthesis reactor assembly may especially be formed by a water-cooled and/or gas-cooled reactor.

The molar flow present in the gas phase in the synthesis reactor assembly may decrease on account of the stoichiometry of the methanol formation reaction. This means the “number” of molecules in the gas phase decreases as a result of the reaction, thus reducing the pressure. A reduced molar flow in the gas phase can reduce the pressure in the synthesis loop provided that the amount of substance lost from the gas phase is not replenished with the feed gas entering the synthesis loop.

The molar flow may be reported in kmol per unit time for example.

Due to the stoichiometry of the methanol formation reaction the first reactant in the feed gas to the synthesis loop is preferably present in a lower proportion than the second reactant. It is preferable when a ratio of the first reactant to the second reactant is in the range from one to three with a variance of 5% or from one to two with a variance of 5%. In the present case the term proportion is to be understood as meaning the molar proportion in mol % of the feed gas stream. A ratio of one to three with a variance of 5% is especially preferred in the case of carbon dioxide and hydrogen. A ratio of one to two with a variance of 5% is especially preferred in the case of carbon monoxide and hydrogen.

The synthesis loop preferably comprises the synthesis reactor assembly and all conduits which connect the outlet of the synthesis reactor assembly to the inlet of the synthesis reactor assembly.

The pressure in the synthesis loop may substantially be determined via the amount of substance in the gas phase, the temperature and the synthesis loop volume. The reduction in the molar flow in the gas phase may be a consequence of the reaction of the first reactant and the second reactant to afford methanol in the synthesis reactor assembly and the condensation and separation of the methanol. The more product is formed and separated, the higher the pressure drop of the synthesis loop may be, provided that the amount of substance lost from the gas phase is not replenished with the feed gas.

The first reactant preferably has a higher solubility than the second reactant in a methanol-water mixture and so the proportion of the first reactant in the gas phase may be further reduced by a solubility in this methanol-water mixture.

The condensation of the methanol formed in the synthesis reactor assembly may be effected in two steps. In the first step the product mixture at the outlet of the synthesis reactor assembly is cooled via a heat exchanger in countercurrent operation. At the same time the gas stream at the inlet of the synthesis reactor assembly is heated via the heat exchanger. In the second step in one or more air-or water-cooled heat exchangers the product gas mixture at the outlet of the synthesis reactor assembly, including partially condensed methanol and unreacted reactants, is cooled to the final temperature of the separator, thus condensing further methanol.

The reaction rate of the first reactant and the second reactant to afford methanol may be determined in the synthesis reactor assembly at constant temperature and constant catalyst amount via the partial pressures of the first reactant and the second reactant. Furthermore on account of the equilibrium reaction the reaction rate may also be determined via the partial pressure of the methanol and the partial pressure of one or more byproducts.

An alternating load operation refers to the operation of the synthesis reactor assembly at varying load, i.e. varying reactor inlet gas flow, in particular as a consequence of a varying reactant stream supplied to the synthesis loop. The so-called load range for the reactant stream supplied to the synthesis loop is preferably between 0% and 100% of plant capacity, preferably between 20% and 100% of plant capacity, at any instant in the process. A load of 100% is also referred to as full load. A load range below 100% is also referred to as partial load.

In at least one first case the molar flow rate of the feed gas stream is reduced. In the case of a load change from a higher load range to a lower load range the molar flow rate of the feed gas stream may fall. This entails a fall in the molar flow of the first reactant and/or the molar flow of the second reactant.

Due to the slow response of the gas amount and composition present in the synthesis loop the synthesis reactor assembly initially continues to convert the reactants as in the higher load range. However, with the reduced molar flow rate a deficit of the first reactant and/or the second reactant can occur and thus a pressure drop of the partial pressures of the first reactant and/or the second reactant.

The reduction in the proportion of the first reactant in the feed gas stream at least temporarily reduces the production rate of the methanol. This has the advantage that the partial pressure of the first reactant is reduced, thus impeding the reaction and reducing the production rate of methanol. This has the further advantage that the amount of substance in the gas phase is stabilized and the pressure in the synthesis loop falls only slightly, if at all. Since the production rate of methanol is reduced, less of the first reactant and less of the second reactant are converted. Thus the reduced reactant molar flow rate per unit time causes less methanol to be formed per unit time, with the result that the pressure in the synthesis reactor assembly falls less severely due to the stoichiometry of the methanol formation reaction. The deficit in first or second reactant thus contributes to the stabilization of the pressure in the synthesis reactor assembly.

In a stoichiometric ratio of the reactants the first reactant preferably accounts for a smaller molar proportion in the feed gas stream into the synthesis loop and into the synthesis reactor assembly compared to the second reactant. An inhibition of the reaction can therefore be achieved comparatively simply by reduction of the proportion of the first reactant. This has the advantage that a reduction in the molar proportion based on the total amount of substance of reactants can be minimized, thus effecting a small partial pressure reduction and thus exerting a smaller influence on the total pressure drop.

The described process is especially suitable for reacting to comparatively rapid fluctuations in the feed gas stream. It is also possible to react to slower fluctuations by other means. In order to achieve an advantage over a conventional process it is therefore sufficient that in at least one first case of a reduction in a molar flow rate of the feed gas stream the production rate of the methanol is at least temporarily reduced by reducing a proportion of the first reactant in the feed gas stream. Changing the supply of the first reactant may also be performed even before a load change is initiated, i.e. before the supply of a second reactant (H2 from the electrolysis) changes. The ratio of the reactants in the feed gas stream in the case of an increase in the molar flow rate of the feed gas stream is generally immaterial to the control of the pressure in the synthesis loop. The ratio of the reactants in the feed gas stream at constant feed gas stream is also insignificant. It is sufficient that in at least one first case of an (expected) reduction in a molar flow rate of the feed gas stream a production rate of the methanol is at least temporarily reduced to effect short-acting compensation of the effects of the reduction in the molar flow rate of the feed gas stream. This is because the pressure in the pipe conduit system can also be influenced in other ways in addition to influencing the ratio of the reactants in the feed gas stream. All options that are also employed in a conventional assembly for synthesis of methanol are in particular contemplated in this regard. For example excessive pressure may be released via an overpressure valve which can react relatively quickly. The risk of excessively high pressure is thus qualitatively distinct from the risk of excessively low pressure.

It is also unnecessary for the proportion of the first reactant in the feed gas stream to be reduced every time the molar flow rate of the feed gas stream decreases. It is sufficient when the proportion of the first reactant in the feed gas stream is reduced at least in a first case of a reduction in the molar flow rate of the feed gas stream, i.e. at least once during performance of the process. Especially in the case of a slow reduction in the molar flow rate of the feed gas stream it may be unnecessary to react by reducing the proportion of the first reactant in the feed gas stream. However, it is preferable when the proportion of the first reactant in the feed gas stream is reduced every time the molar flow rate of the feed gas stream falls faster than a pressure change threshold value and/or is reduced every time the molar flow rate of the feed gas stream falls to a value below a pressure threshold value.

In a preferred embodiment in the at least one first case the proportion of the first reactant in the feed gas stream is reduced by adding a buffer gas to the feed gas stream.

The reaction rate of the reactants to afford methanol can be reduced by reducing the partial pressure of the reactants. The probability of the reaction of the first reactant and the second reactant to afford methanol decreases at lower partial pressure. Adding a buffer gas to the feed gas stream at a defined total pressure reduces the proportion of the reactants in the feed gas stream and thus their partial pressure.

A reduction in the production rate is advantageous since the first reactant and the second reactant react to afford methanol and the reacted amount of substance is withdrawn from the gas phase of the synthesis loop, thus favouring a pressure drop. If less methanol is produced, correspondingly less reactant is consumed.

This embodiment is preferably used to reduce the partial pressure of the first reactant and the second reactant, to inhibit the reaction and to produce less methanol. A further advantage of this embodiment is that the buffer gas can stabilize a pressure drop by replenishing an amount of substance of the gas phase missing in the synthesis loop for pressure maintenance, thus stabilizing the total pressure in the synthesis loop.

In a further preferred embodiment in the at least one first case the proportion of the first reactant in the feed gas stream is reduced by reducing an amount of the first reactant in the feed gas stream relative to an amount of the second reactant in the feed gas stream.

This can preferably be achieved by adding the second reactant to the feed gas stream in a superstoichiometric amount.

The amount of the second reactant preferably fluctuates. A fluctuation is to be understood as meaning a change in the molar flow and/or the partial pressure of the second reactant over time.

This embodiment is preferably used to reduce the production rate of the methanol by reducing the partial pressure of the first reactant to such an extent that the reaction is inhibited.

In a first option the molar flow rate of the second reactant can be increased. This makes it possible to inhibit the reaction by creating a deficit of first reactant, the excess of second reactant compensating the deficit of the amount of substance of a first reactant. This makes it possible to stabilize the pressure in the synthesis loop. The disadvantage of this option is that the second reactant is not readily available. This is because in the at least one first case the feed gas stream may be reduced especially when the second reactant has temporarily reduced availability.

In a second option the molar flow rate of the first reactant is reduced before the second reactant falls due to fluctuation. This has the effect that the second reactant is present in the feed gas stream in a superstoichiometric amount. By reducing the amount of the first reactant the diminishing amount of the first reactant reacts with the second reactant to afford methanol, thus reducing the proportion of the first reactant in the synthesis loop. This reduces the partial pressure of the first reactant and the reaction is inhibited. The content of a second reactant in the synthesis loop is simultaneously enriched. If less second reactant is now available due to the occurrence of fluctuation the feed gas stream may be reduced and the pressure in the synthesis loop is stable on account of the inhibited reaction.

The proportion of the first reactant in the feed gas stream in the at least one first case can be reduced to such an extent that the first reactant in the synthesis loop is present in a substoichiometric amount.

The preceding embodiments have in common that in at least one first case a reduction of a molar flow rate of the feed gas stream reduces a production rate of the methanol in that the proportion of the first reactant inhibits the reaction to afford methanol due to a low partial pressure.

In a further preferred embodiment methanol from an outlet stream of the synthesis reactor assembly is separated and the remaining outlet stream of the synthesis reactor assembly is at least partially returned as a recycle gas stream, wherein in the at least one first case a ratio of a molar flow rate of the recycle gas stream to a molar flow rate of the feed gas stream is increased.

The ratio of the molar flow rate of the recycle gas stream to the molar flow rate of the feed gas stream is also referred to as the recycle ratio.

It is preferable when a recycle ratio of 2 to 200 is present in the at least one first case. A recycle ratio of 3 to 30 is preferred.

It is preferable when the mixed feed gas stream and recycle gas stream at the inlet of the synthesis reactor assembly have a molar ratio of the first reactant to the second reactant in the range from 0.001 to 0.33, preferably in the range from 0.005 to 0.33.

A significant increasing of the recycle ratio and thus elevated enrichment of unreacted components is generally only possible in the partial load range since the maximum recycle ratio is limited by the upper limit of a conveyable recycle gas stream.

This embodiment is preferably used to increase the conversion of the reactants and accumulate inert gases and unreacted reactants as a buffer.

The first reactant and the second reactant may be mixed in the inlet of the synthesis reactor assembly and/or upstream of the inlet of the synthesis reactor assembly. Further streams such as the recycle gas stream and the buffer gas may be admixed with the feed gas stream in the inlet and/or upstream of the inlet of the synthesis reactor assembly.

In at least one second case the molar flow rate of the feed gas stream is preferably increased. Analogously to the at least one first case it is unnecessary for the proportion of the first reactant in the feed gas stream to be increased every time the molar flow rate of the feed gas stream increases. It is sufficient when the proportion of the first reactant in the feed gas stream is increased in at least one second case of an increase in the molar flow rate of the feed gas stream, i.e. at least once during performance of the process.

In a further preferred embodiment in at least one second case of an increase in a molar flow rate of the feed gas stream a production rate of the methanol is at least temporarily increased by increasing the proportion of the first reactant in the feed gas stream.

Any pressure increase in the synthesis loop is preferably released by increasing the molar flow rate via a purge gas conduit. It is particularly preferable when the outlet stream of the synthesis reactor assembly is at least partially removed as purge gas. This makes it possible to remove the second reactant which is present in the synthesis loop in a superstoichiometric amount and/or the buffer gas from the synthesis loop. The partial pressure of the first reactant is increased with an amount of first reactant newly introduced into the loop and no longer inhibits the reaction. The elevated molar flow rate of the feed gas stream then correspondingly increases the production rate of the methanol.

This embodiment is preferably used to rapidly increase the production rate of the methanol in the at least one second case and to prevent the total pressure in the synthesis reactor assembly from rising.

In a further preferred embodiment in the at least one second case the proportion of the first reactant in the feed gas stream is increased by increasing an amount of the first reactant in the feed gas stream relative to an amount of the second reactant in the feed gas stream.

The first case of a reduction in the molar flow rate may cause the second reactant to be present in the synthesis loop in a superstoichiometric amount.

When the feed gas stream increases rapidly the production rate of the methanol may initially be too low. This may result in increasing pressure in the synthesis loop. To increase the production rate of the methanol again the proportion of the first reactant in the feed gas stream is at least temporarily increased. The partial pressure of the first reactant increases and no longer inhibits the production rate of the methanol. This elevated proportion of the first reactant can react with the second reactant present in a superstoichiometric amount and increase the production rate of the methanol so that the first reactant and the second reactant react to afford methanol. This has the effect that the excess molar flow is reduced by the reaction of the first reactant and the second reactant to afford methanol as a result of the elevated feed gas stream.

This embodiment is preferably used to increase the production rate of the methanol in the at least one second case and to prevent the total pressure in the synthesis loop from rising.

The first reactant is preferably carbon dioxide or carbon monoxide.

The second reactant is preferably hydrogen.

The second reactant particularly preferably derives from a water electrolysis with renewable energy.

The feed gas stream by preference comprises 0 mol % to 35 mol % of carbon dioxide and 65 mol % to 100 mol % of hydrogen. The feed gas stream preferably comprises 15 mol % to 30 mol % of carbon dioxide and 70 mol % to 85 mol % of hydrogen.

In a further preferred embodiment the buffer gas contains an inert gas or a plurality of inert gases and/or methane and/or hydrogen.

The inert gas is preferably argon. The inert gas is particularly preferably nitrogen. It is also possible to mix two or more inert gases. The inert gas preferably comprises a mixture of argon and/or nitrogen.

In a further preferred embodiment a total pressure in the synthesis reactor assembly fluctuates by not more than 40%, preferably not more than 20%, preferably not more than 10%, during performance of the process.

A desired total pressure is an operating pressure of the synthesis reactor assembly independent of the load range. High pressure fluctuations increase the demands on the employed pipe conduits and apparatuses since these must be configured for pressure fluctuations.

The described process is useful particularly when the intention is to allow synthesis of methanol in alternating load operation, especially when the feed gas stream is to be reduced. This case can occur when the second reactant fluctuates. Instead of completely replenishing the reduced reactant molar flow via a buffer gas to prevent a pressure drop the reaction to afford methanol may be inhibited by reducing the first reactant molar flow. It is particularly advantageous to reduce the first reactant molar flow before the second reactant molar flow is reduced by fluctuation.

A further aspect of the invention provides an apparatus for synthesis of methanol which comprises a synthesis reactor assembly, a first reactant gas source for a first reactant, a second reactant gas source for a second reactant and a control means, wherein the first reactant gas source and the second reactant gas source are connected to an inlet of the synthesis reactor assembly and wherein the control means is adapted for operating the apparatus with the described process.

The first reactant gas source may preferably be a carbon dioxide separation process or a carbon dioxide conduit. The first reactant is preferably carbon dioxide and/or carbon monoxide.

The second reactant gas source is preferably a water electrolysis process. The water electrolysis process is preferably operated with a supply of renewable energy which may fluctuate. The second reactant is preferably hydrogen.

For the control means it is preferable to measure the gas composition, the molar flow and the pressure at various points. Especially in the feed gas stream it is preferable to investigate the proportion of the first reactant, the second reactant and any residual components such as for example inert gases that are present before entry into the synthesis loop. Further measurement points may be provided at the inlet of the synthesis reactor assembly or at the outlet of the synthesis reactor assembly. If a recycle gas stream is present the gas composition, the molar flow and the pressure may be measured in the recycle conduit.

In a further preferred embodiment the apparatus further comprises a buffer gas source for a buffer gas, wherein the buffer gas source is connected to an inlet of the synthesis reactor assembly.

This embodiment is preferably used in the at least one first case to reduce the proportion of the first reactant in the feed gas stream by adding a buffer gas to the feed gas stream.

In a further preferred embodiment the apparatus further comprises a recycle conduit by means of which an outlet of the synthesis reactor assembly is connected to an inlet of the synthesis reactor assembly.

In this preferred embodiment the conversion of the reactants to afford methanol may be increased and inert gases and also unreacted reactants accumulated as buffer.

In another preferred embodiment the apparatus further comprises a purge gas conduit which branches off from the recycle conduit.

In this preferred embodiment an elevated pressure in the synthesis loop may be maintained in a defined range/reduced in a controlled manner as a reaction to a feed gas stream that has spontaneously increased.

The use of an apparatus configured as described has the advantage of enabling alternating load operation in the synthesis of methanol. Alternating load operation can occur through reduction and increasing of the feed gas stream and be based on a fluctuating second reactant. Reduction and increasing of the proportion of the first reactant in the feed gas stream makes it possible to control the reaction rate and thus control the total pressure in the synthesis loop.

### BRIEF DESCRIPTION OF THE DRAWINGS

The invention will now be more particularly described with reference to the figure. The figure shows a particularly preferred exemplary embodiment but the invention is not limited thereto. The figure and the relative sizes shown therein are only schematic. In the figures:

FIG. 1 shows a schematic representation of an apparatus according to the invention for synthesis of methanol.

### DETAILED DESCRIPTION OF THE PREFERRED EMBODIMENTS

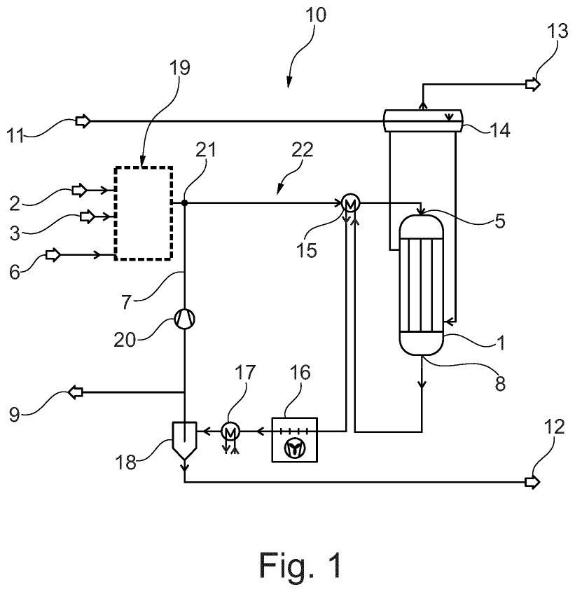

FIG. 1 shows a schematic representation of an apparatus 10 according to the invention for synthesis of methanol which comprises a synthesis reactor assembly 1, a first reactant gas source 2 for a first reactant, a second reactant gas source 3 for a second reactant, a buffer gas source 6 for a buffer gas and a control means (not shown), wherein the first reactant gas source 2, the second reactant gas source 3 and the buffer gas source 6 are connected to an inlet 5 of the synthesis reactor assembly 1. The first reactant, the second reactant and the buffer gas, provided a buffer gas is introduced, are compressed via a first compressor 19. The first reactant is carbon dioxide. The second reactant is hydrogen deriving from an electrolysis process with renewable energy.

The apparatus 10 further comprises a recycle conduit 7 by means of which an outlet 8 of the synthesis reactor assembly 1 is connected to the inlet 5 of the synthesis reactor assembly 1. The recycling conduit 7 comprises a heat exchanger 15, a synthesis air cooler 16, a final water cooler 17 and a separator 18. A synthesis loop 22 comprises the synthesis reactor assembly 1, the recycle conduit 7 and a conduit from a mixing point 21 to the inlet 5 of the synthesis reactor assembly 1.

The synthesis reactor assembly 1 produces methanol and steam from carbon dioxide and hydrogen. The condensation of the methanol formed in the synthesis reactor assembly 1 is carried out in two steps. In the first step the product mixture at the outlet of the synthesis reactor assembly 1 is cooled via the heat exchanger 15 in countercurrent operation. The gas stream of the synthesis loop 22 is simultaneously heated. The synthesis loop stream contains the feed gas stream and the recycle gas stream. In the second step the product gas mixture at the outlet of the synthesis reactor assembly 1 and the partially condensed methanol and the unreacted reactants are cooled to the final separation temperature in the synthesis air cooler 16 and in the final water cooler 17. Liquid methanol is separated from the gas stream in the separator 18. The methanol is discharged at a methanol outlet 12.

A purge gas conduit 9 branches off from the recycle conduit 7. The recycle gas stream in the recycle conduit 7 is compressed to an operating pressure in a second compressor 20. The feed gas stream and the buffer gas stream are mixed with the recycle gas stream in the mixing point 21.

A fresh water source 11 provides water to a steam boiler 14. This produces steam 13 and hot water and/or saturated steam. The temperature of the synthesis reactor assembly 1 can be kept constant during the fluctuation of the feed gas stream either by cooling the reaction heat and evaporating water from the shell side of the synthesis reactor assembly 1 or in the case of insufficient waste reaction heat by heating through additional supply of saturated steam or hot water on the shell side from the steam boiler 14.

The control means is adapted for operating the apparatus 10 with the following process for synthesis of methanol.

A feed gas stream comprises carbon dioxide and hydrogen and is supplied to the synthesis reactor assembly 1, wherein the synthesis reactor assembly 1 uses carbon dioxide and hydrogen to produce methanol, wherein in at least one first case of a reduction in a molar flow rate of the feed gas stream a production rate of the methanol is temporarily reduced by reducing a proportion of carbon dioxide in the feed gas stream.

In the at least one first case the proportion of carbon dioxide in the feed gas stream is reduced by adding a buffer gas composed of nitrogen and hydrogen to the feed gas stream. Furthermore in the at least one first case the proportion of carbon dioxide in the feed gas stream is reduced by reducing an amount of carbon dioxide in the feed gas stream relative to an amount of hydrogen in the feed gas stream. The proportion of carbon dioxide in the feed gas stream is reduced such that the carbon dioxide in the feed gas stream is present in a substoichiometric amount.

The methanol from an outlet stream from an outlet 8 of the synthesis reactor assembly 1 is separated and the remaining outlet stream of the synthesis reactor assembly 1 is partially returned as a recycle gas stream, wherein in the at least one first case a ratio of a molar flow rate of the recycle gas stream to a molar flow rate of the feed gas stream is increased. In the at least one first case the recycle ratio is for example 20.

In at least one second case of an increase in a molar flow rate of the feed gas stream the production rate of the methanol is temporarily increased by increasing the proportion of the carbon dioxide in the feed gas stream. To this end the outlet stream of the synthesis reactor assembly 1 is partially removed as purge gas.

The proportion of carbon dioxide in the feed gas stream is increased by increasing an amount of carbon dioxide in the feed gas stream relative to an amount of hydrogen in the feed gas stream.

The total pressure in the synthesis reactor assembly 1 fluctuates by not more than 10% during performance of the process.

### REFERENCE NUMERALS

1 Synthesis reactor assembly
2 First reactant gas source
3 Second reactant gas source
5 Inlet of synthesis reactor assembly
6 Buffer gas source
7 Recycle conduit
8 Outlet of synthesis reactor assembly
9 Purge gas conduit
10 Apparatus
11 Fresh water source
12 Methanol outlet
13 Steam
14 Steam boiler
15 Heat exchanger
16 Synthesis air cooler
17 Water cooler
18 Separator
19 First compressor
20 Second compressor
21 Mixing point
22 Synthesis loop

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

1.-15. (canceled)

16. A process for synthesis of methanol, comprising:
introducing a feed gas stream comprising at least a first reactant and a second reactant into a synthesis reactor assembly,
wherein the synthesis reactor assembly produces methanol with the first reactant and the second reactant,
wherein in at least one first case of a reduction in a molar flow rate of the feed gas stream a production rate of the methanol is at least temporarily reduced by reducing a proportion of the first reactant in the feed gas stream.

17. The process according to claim 16, wherein in the at least one first case the proportion of the first reactant in the feed gas stream is reduced by adding a buffer gas to the feed gas stream.

18. The process according to claim 16, wherein in the at least one first case the proportion of the first reactant in the feed gas stream is reduced by reducing an amount of the first reactant in the feed gas stream relative to an amount of the second reactant in the feed gas stream.

19. The process according to claim 16, wherein in the at least one first case a proportion of the first reactant in the feed gas stream is reduced so that the first reactant is present in the feed gas stream in a substoichiometric amount.

20. The process according to claim 16, wherein methanol from an outlet stream of the synthesis reactor assembly is separated and the remaining outlet stream of the synthesis reactor assembly is at least partially returned as a recycle gas stream and wherein in the at least one first case a ratio of a molar flow rate of the recycle gas stream to a molar flow rate of the feed gas stream is increased.

21. The process according to claim 16, wherein in at least one second case of an increase in a molar flow rate of the feed gas stream a production rate of the methanol is at least temporarily increased by increasing the proportion of the first reactant in the feed gas stream.

22. The process according to claim 21, wherein in the at least one second case the proportion of the first reactant in the feed gas stream is increased by increasing an amount of the first reactant in the feed gas stream relative to an amount of the second reactant in the feed gas stream.

23. The process according to claim 16, wherein the first reactant is carbon dioxide and/or carbon monoxide.

24. The process according to claim 16, wherein the second reactant is hydrogen.

25. The process according to claim 24, wherein the second reactant derives from a water electrolysis with renewable energy.

26. The process according to claim 17, wherein the buffer gas contains an inert gas or a plurality of inert gases and/or methane and/or hydrogen.

27. The process according claim 16, wherein a total pressure in the synthesis reactor assembly fluctuates by not more than 40% during performance of the process.

28. An apparatus for synthesis of methanol which comprises a synthesis reactor assembly, a first reactant gas source for a first reactant, a second reactant gas source for a second reactant and a control means, wherein the first reactant gas source and the second reactant gas source are connected to an inlet of the synthesis reactor assembly and wherein the control means is adapted for operating the apparatus with a process according to claim 16.

29. The apparatus according to claim 28, further comprising a buffer gas source for a buffer gas, wherein the buffer gas source is connected to an inlet of the synthesis reactor assembly.

30. The apparatus according to claim 28, wherein the apparatus further comprises a recycle conduit by means of which an outlet of the synthesis reactor assembly is connected to an inlet of the synthesis reactor assembly.
